(19) United States
(12) Patent Application Publication
(10) Pub. No.: US 2006/0057576 A1
(43) Pub. Date: Mar. 16, 2006

(54) Microcapillary hybridization chamber

(76) Inventors: Jerzy Paszkowski, Nenzlingen (CH); Andras Guttman, San Diego, CA (US); Xun Wang, San Diego, CA (US)

Correspondence Address: JENKINS, WILSON & TAYLOR, P. A., 3100 TOWER BLVD, SUITE 1400, DURHAM, NC, 27707, US

(21) Appl. No.: 10/507,167

(22) PCT Filed: Mar. 12, 2003
(86) PCT No.: PCT/US03/07688

§ 371 Date: Jul. 18, 2005

Related U.S. Application Data
(60) Provisional application No. 60/363,869, filed on Mar. 12, 2002.

(51) Int. Cl. C12Q 1/68; C12M 1/34

(52) U.S. Cl. 435/6; 435/287.2; 204/451

(57) ABSTRACT

The invention provides a microcapillay hybridization chamber made of a narrow bore tubing with probe segments. Each probe segment has oligonucleotide probes covalently attached to the inner wall of the tubing and the oligonucleotide probes within each segment have identical, known sequences. Many oligonucleotide probe segments can be present within a single centimeter of tubing. The invention further provides methods for using the microcapillary hybridization chambers in hybridization assays.

### FIELD OF THE INVENTION

This invention relates to a capillary hybridization chamber having a multitude of immobilized, spatially-separated nucleic acid probes.

### BACKGROUND OF THE INVENTION

Substrate-bound oligonucleotide arrays, such as Affymetrix probe arrays, can be used to hybridize a target nucleic acid to many thousands of different oligonucleotide probe types. More than 500 different probe types can be present per each square centimeter. Each probe type has a discrete, known location on the array. These arrays are useful for determining the sequence of nucleic acid molecules in a target, and for discriminating between genetic variants that can differ in sequence by just a few nucleotides or even by just one nucleotide. Probe arrays are typically formed as two-dimensional arrays on a glass or silicon substrate. These substrates are fragile, so they must be handled with care and may have to be kept in a protective container.

Patent application Ser. No. 08/512,027 entitled “Method and Apparatus for Producing Position-Addressable Combinatorial Libraries,” filed Aug. 7, 1995, discloses a one-dimensional position-addressable array of oligonucleotides on a filament. However, the probes are placed on the outside of the filament and are therefore exposed to the air and vulnerable to physical injury and chemical degradation.

Therefore, position-addressable probe arrays contained within a chamber that not only shelters the probes from physical and chemical degradation but permits repeated, low volume hybridization reactions and ready detection of hybridized target nucleic acids would be very useful for streamlining and generating large amounts of data in hybridization assays.

### SUMMARY OF THE INVENTION

The invention provides microcapillary hybridization chambers that include a narrow bore capillary tubing with a one dimensional array of probes at precisely predetermined positions along the tubing. The microcapillary tubing serves as a low-volume hybridization chamber that can be readily flushed with solutions to prepare the microcapillary hybridization chamber for hybridization, to introduce solutions of target DNA and to wash any unhybridized target DNA from the chamber. After hybridization and washing, the microcapillary hybridization chambers can be fed through or introduced into instrumentation capable of detecting target-probe hybrids. The microcapillary hybridization chambers are robust, easily manipulated and reusable.

Accordingly, this invention provides a microcapillary hybridization chamber that includes a capillary tube having a distinguishable segments, each segment comprising a set of oligonucleotides of defined sequence, each oligonucleotide immobilized to the capillary tube within a defined segment. Oligonucleotides immobilized within the microcapillary hybridization chamber are generally single-stranded before use in hybridization assays, and can hybridize to complementary nucleic acids. Therefore, oligonucleotides immobilized within the microcapillary hybridization chamber can act as probes for target nucleic acids of interest.

Hybridization of a target nucleic acid to a complementary oligonucleotide immobilized within the microcapillary hybridization chamber can be detected by any method available to one of skill in the art. For example, the mixture of target nucleic acids can be labeled with reporter molecules. Alternatively, reagents that bind only to double-stranded hybrids can be used. After hybridization the reagent binds to the double-stranded hybrids and can be directly detected through an attached reporter molecule, or indirectly detected by using a secondary reporter molecule that selectively binds to the reagent.

Oligonucleotides with different, known sequences, are located at discrete, known segment locations within the microcapillary tubing of the hybridization chamber. The length of each oligonucleotide segment need only be large enough to distinguish hybridization of target nucleic acids in one segment from hybridization of target nucleic acids in another segment. Hence, for example, there can be about 50 oligonucleotide segments per centimeter to about one million oligonucleotide segments per centimeter. Preferably, there are at least about 100, more preferably 200, oligonucleotide segments per centimeter. Even more preferably, there are at least about 500 oligonucleotide segments per centimeter. Most preferably, there are at least about 1000 oligonucleotide segments per centimeter. However, in some especially preferred embodiments there are at least about ten thousand, or even about one hundred thousand, oligonucleotide segments per centimeter.

The capillary tubing of the microcapillary hybridization chamber can be any length that is conveniently manipulated by one of skill in the art, for example, about one millimeter to about ten meters. Preferred lengths are about 1 millimeter to about 100 meters.

In yet another aspect of the present invention, a microcapillary hybridization chamber is provided that includes a narrow bore tubing with probe segments attached to the inner wall of the tubing at predefined positions, wherein each probe segment includes identical probes. In accordance with at least some embodiments of the present invention each probe segment is selected from deoxyribonucleic acids, ribonucleic acids, synthetic oligonucleotides, antibodies, proteins, peptides, lectins, modified polysaccharides, cells, synthetic composite macromolecules, functionalized nanostructures, synthetic polymers, modified/blocked nucleotides/nucleosides, modified/blocked amino acids, flurophores, chromophores, ligands, chelates, hapten, and combinations thereof.

In a further aspect of the present invention, a method is provided for performing a hybridization assay between at least one target and a microcapillary hybridization chamber, the method includes: introducing a sample comprising said at lest one target into said microcapillary hybridization chamber; applying an electrical potential to said microcapillary hybridization chamber with said at least one target; washing unhybridized said at least one target out of said microcapillary hybridization chamber; and detecting hybridization. The microcapillary hybridization chamber includes a narrow bore tubing with probe segments attached to the inner wall of the tubing at predefined positions, wherein each probe segment has identical probes.

In another aspect of the present invention, a method is provided for making a microcapillary hybridization chamber that includes: providing a substrate wall, wherein said substrate wall is internal to a narrow bore capillary tube and attaching a plurality of probe segments to said substrate; wherein each of said plurality of probe segments are spatially discrete from each other.

In a further aspect of the present invention, a method is provided for controlling the stringency in a microcapillary hybridization chamber that includes applying an electrical potential to each probe segment in said microcapillary hybridization chamber, wherein the microcapillary hybridization chamber includes a narrow bore tubing with said probe segments attached to the inner wall of the tubing at predefined positions, wherein each probe segment has identical probes. According to at least one embodiment in accordance with the present invention the method further includes detecting the state of hybridization in the microcapillary hybridization chamber. In a further embodiment in accordance with the present embodiment each electrical potential is adjusted until mis-matched hybridizations are eliminated.

In another aspect of the present invention an apparatus is provided for detecting hybridization in a microcapillary hybridization chamber that includes a microcapillary hybridization chamber, wherein the microcapillary hybridization chamber includes a narrow bore tubing with probe segments attached to the inner wall of the tubing at predefined positions, wherein each probe segment has identical probes; a detector for detecting hybridization signals in said microcapillary hybridization chamber; and a computer system operationally coupled to said detector, the computer system includes a program that executes instructions for displaying the detected hybridization signals. In at one embodiment in accordance with the prevent invention the detector includes excitation optics for focusing excitation light on at least one of the probe segments. In another embodiment in accordance with the present invention the program further includes instructions for executing determining fluorescent intensity; removing data outliers; and calculating the relative binding affinity of said hybridization signals. In a further embodiment in accordance with the present invention the program further includes instructions for displaying an image of probe segment colors based on at least one of light emission or binding affinity.

Other features and advantages will become apparent from the following detailed description, drawings, and claims.

### DESCRIPTION OF THE DRAWING

The invention is pointed out with particularity in the appended claims. The advantages of the invention described above, as well as further advantages of the invention, are better understood by reference to the following detailed description taken in conjunction with the accompanying drawings, in which:

FIG. 1 provides a drawing of a microcapillary hybridization chamber in accordance with at least some of the embodiments of the present invention.

FIG. 2 provides a schematic diagram of a closer view of just a few oligonucleotide segments within the interior wall of the microcapillary tube, showing how hybridization may occur between one oligonucleotide probe and a complementary target nucleic acid in accordance with at least some of the embodiments of the present invention.

FIG. 3 provides a schematic diagram of a prior art two-dimensional probe array, such as a chip, where target nucleic acids have hybridized to particular probes 3.

### DETAILED DESCRIPTION OF THE INVENTION

Nucleic acid probes or oligonucleotides are introduced and immobilized at discrete locations or segments within a narrow bore capillary tube to generate a microcapillary hybridization chamber. The invention provides such microcapillary hybridization chambers and methods for using these microcapillary hybridization chambers.

Definitions

The following terms are intended to have the following general meanings as they are used herein.

“Complementary” or “complementarity” are used to define the degree of base-pairing or hybridization between nucleic acids. For example, as is known to one of skill in the art, adenine (A) can form hydrogen bonds or base pair with thymine (T) and guanine (G) can form hydrogen bonds or base pair with cytosine (C). Hence, A is complementary to T and G is complementary to C. A nucleic acid strand that has a complementary base at every position relative to a second nucleic acid strand is the complement of the second nucleic acid strand. Complementarity may be complete when all bases in a double-stranded nucleic acid are base paired. Alternatively, complementarity may be “partial,” in which only some of the bases in a nucleic acid are matched according to the base pairing rules. The degree of complementarity between nucleic acid strands has an effect on the efficiency and strength of hybridization between nucleic acid strands.

“Hybridization” refers to the process of annealing complementary nucleic acid strands by forming hydrogen bonds between nucleotide bases on the complementary nucleic acid strands. Hybridization, and the strength of the association between the nucleic acids, is impacted by such factors such as the length of the hybridizing nucleic acids, the degree of complementary between the hybridizing nucleic acids, the stringency of the conditions involved, the melting temperature (T.) of the formed hybrid, and the G:C ratio within the nucleic acids.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid.

The term “oligonucleotide” as used herein is defined as a molecule comprised of two or more deoxyribonucleotides or ribonucleotides, preferably more than three, and usually more than ten. There is no precise upper limit on the size of an oligonucleotide. However, in general, an oligonucleotide is shorter than about 250 nucleotides, preferably shorter than about 200 nucleotides and more preferably shorter than about 100 nucleotides. The exact size will depend on many factors, which in turn depends on the ultimate function or use of the oligonucleotide. When used as probes immobilized to the present microcapillary hybridization chambers, oligonucleotides are about six to about fifty nucleotides in length, preferably about eight to about thirty nucleotides in length and more preferably about ten to about twenty nucleotides in length. The most preferred size for oligonucleotide probes of the invention is about twelve to about nineteen nucleotides in length. Oligonucleotide can be made or isolated by any procedure available to one of skill in the art, including chemical synthesis, DNA replication, reverse transcription, or a combination thereof.

A “probe” is an oligonucleotide that can bind a particular target nucleic acid. Depending on context, the term “probe” refers both to individual oligonucleotide molecules and to the collection of same-sequence oligonucleotide molecules surface-immobilized at a discrete location in the microcapillary hybridization chambers of the invention. However, probe is not limited to oligonucleotide and can include, for example, deoxyribonucleic acids (DNA), ribonucleic acids (RNA), synthetic oligonucleotides, antibodies, proteins, peptides, lectins, modified polysaccharides, cells, synthetic composite macromolecules, functionalized nanostructures, synthetic polymers, modified/blocked nucleotides/nucleosides, modified/blocked amino acids, flurophores, chromophores, ligands, chelates and haptens.

A “target” nucleic acid is at least partially complementary to an oligonucleotide or probe of the invention. Target nucleic acids may be naturally-occurring or man-made nucleic acid molecules. Also, they can be present in test samples obtained from any source and may be employed in their unaltered state or after labeling with a reporter molecule. However, target is not limited to oligonucleotide and can include, for example, deoxyribonucleic acids (DNA), ribonucleic acids (RNA), synthetic oligonucleotides, antibodies, proteins, peptides, lectins, modified polysaccharides, cells, synthetic composite macromolecules, functionalized nanostructures, synthetic polymers, modified/blocked nucleotides/nucleosides, modified/blocked amino acids, flurophores, chromophores, ligands, chelates and haptens.

By “specific binding entity” is generally meant a biological or synthetic molecule that has specific affinity to another molecule, macromolecule or cells, through covalent bonding or non-covalent bonding. Specific binding entities include, but are not limited to: deoxyribonucleic acids (DNA), ribonucleic acids (RNA), synthetic oligonucleotides, antibodies, proteins, peptides, lectins, modified polysaccharides, cells, synthetic composite macromolecules, functionalized nanostructures, synthetic polymers, modified/blocked nucleotides/nucleosides, modified/blocked amino acids, flurophores, chromophores, ligands, chelates and haptens.

By “stringency control” is generally meant the ability to discriminate specific and non-specific binding interactions by changing some physical or chemical parameter. In the case of nucleic acid hybridizations, for example, temperature control and/or electric potential can be used for controlling the stringency. Stringency control is used for controlling hybridization specificity, and is particularly important for resolving one base mis-matches in point mutations. Stringency control can also be applied to multiple-base mis-match analysis.

Microcapillary Hybridization Chambers

The microcapillary hybridization chambers of the invention can be made from any available narrow bore capillary tubing that can be adapted for immobilization of oligonucleotide probes and that is structurally and chemically impervious to hybridization conditions. Hybridization conditions include temperatures, pre-hybridization, hybridization and washing solutions used by one of skill in the art during hybridization procedures. Preferably, the microcapillary hybridization chambers of the invention are made of a material that permits transmission and detection of a signal (e.g., light, radioactivity, fluorescence, etc.) from a label within the chamber to a detector outside of the chamber.

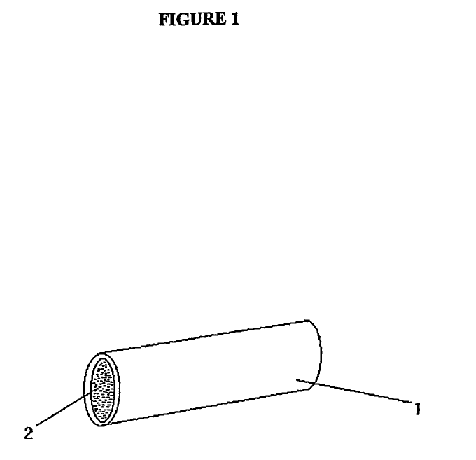

FIG. 1 provides a three-quarter illustration of a microcapillary hybridization chamber in accordance with at least some of the embodiments of the present invention. The microcapillary hybridization chamber has two longitudinal surfaces. The outer surface 1 includes no probe segments. The outer surface 1 is typically clear so as to allow visual or other hybridization detection access to the probes on the inner surface 2. The inner surface or interior bore region 2 includes the probe segments for the hybridization analysis. Probe segments are indicated by the stipple marks on the inner surface 2 of the microcapillary hybridization chamber. The number, density and location of the probe segments can vary.

The material used for microcapillary hybridization chambers can be any material that can be formed into a narrow bore capillary tube and that has the properties described above. The material is preferably optically transparent or translucent to permit detection of the reporter molecule(s) that indicate hybridization has occurred. For example, the material employed can be glass, functionalized glass, Ge, GaAs, GaP, SiO₂, SiN₄, silicon, modified silicon, or any one of a wide variety of gels or polymers such as polypropylene or chlorinated polypropylene, (poly)tetra-fluoroethylene, (poly)vinylidenedifluoride, polystyrene, chloromethylated polystyrene, polycarbonate, or combinations thereof. Other materials will be readily apparent to those skilled in the art upon review of this disclosure.

The inner wall 2 of the narrow bore capillary tubing for use in the microcapillary hybridization chambers of the invention is usually, though not always, composed of the same material as the substrate. Thus, the inner wall 2 can be derivatized or coated with any material simply by allowing the coating material or derivatizing reagent to flow through the narrow bore capillary tubing. For example, the surface of the inner wall 2 of the microcapillary hybridization chamber may be composed of any of a wide variety of materials, for example, polymers, plastics, resins, polysaccharides, silica or silica-based materials, carbon, metals, inorganic glasses, membranes, or any of the above-listed substrate materials. In one embodiment in accordance with the present invention, the inner wall surface 2 will be optically transparent and will have surface Si—OH functionalities, such as those found on silica surfaces.

The wall thickness of the capillary tubing is sufficient to provide structural strength and stability but not so thick as to undermine the detection and localization of signal from a label or reporter molecule attached to a target or hybrid within a discrete segment of the microcapillary hybridization chamber. Materials that are transparent to light are particularly useful when the label is a fluorescent dye, or optical detection is used.

The oligonucleotide probes can be attached to discrete locations within the microcapillary chambers of the invention by any procedure known to one of skill in the art. For example, an oligonucleotide probe of the invention can be attached directly to the substrate of the microcapillary hybridization chamber or to a linker attached to the inner wall of the microcapillary chamber. The linker can, for example, be a long chain bifunctional reagent such as a diol, diamine, ethylene glycol oligomer or amine-terminated ethylene glycol oligomer.

Displacers can be used to prevent mixing and/or diffusion between segments of the microcapillary chamber of reagents used for oligonucleotide synthesis. Preferably, spatially distinct oligonucleotide segments within the microcapillary hybridization chambers are produced through spatially directed oligonucleotide synthesis. As used herein, “spatially directed oligonucleotide synthesis” refers to any method of directing the synthesis of an oligonucleotide to a specific location on the substrate of the microcapillary chamber. Methods for spatially directed oligonucleotide synthesis include, without limitation, light-directed oligonucleotide synthesis, microlithography, application by ink jet, microchannel deposition to specific locations and sequestration with physical barriers. In general these methods involve generating active sites, usually by removing protective groups; and coupling to the active site a nucleotide that can also have a protected active site to facilitate further nucleotide coupling in that segment or other segments.

In one embodiment substrate-bound oligonucleotide arrays are synthesized at specific locations by light-directed oligonucleotide synthesis. Methods for performing such syntheses are disclosed in U.S. Pat. No. 5,143,854, U.S. Pat. No. 5,571,639, and WO0010092. For example, the surface of a solid support can have attached linkers that have photolabile protecting groups at the position to which the oligonucleotide or nucleotide is to be added. The surface of a solid support is illuminated through a photolithographic mask, to produce reactive hydroxyl groups from the photolabile protecting groups in the illuminated regions. The surface is then contacted with a 3′-O-phosphoramidite-activated deoxynucleoside (protected at the 5′-hydroxyl with a photolabile group) and coupling occurs at sites that were exposed to light. The unreacted active sites can be optionally capped. Then, the substrate is rinsed and the surface is illuminated through a second mask. Such illumination activates the photolabile group on the 5′-hydroxyl of the previously added deoxynucleoside to expose an additional hydroxyl group for coupling. A second 5′-protected, 3′-O-phosphoramidite-activated deoxynucleoside (C—X) is presented to the surface. The selective photodeprotection and coupling cycles are repeated until the desired set of products is obtained. Photolabile groups are then optionally removed and the sequence is, thereafter, optionally capped. Side chain protective groups, if present, are also removed. Since photolithography is used, the process can be miniaturized to generate a high-density of oligonucleotide probes within each segment.

This general process can be modified. For example, the nucleotides can be natural nucleotides, chemically modified nucleotides or nucleotide analogs, as long as they have activated hydroxyl groups compatible with the linking chemistry. The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-O-phosphoramidite-activated deoxynucleoside. In this case, the oligonucleotide is synthesized in the 5′ to 3′ direction, that results in a free 5′ end.

The general process of removing protective groups by exposure to light, coupling nucleotides (optionally competent for further coupling) to the exposed active sites, and optionally capping unreacted sites is referred to herein as “light-directed nucleotide coupling.” Tiling strategies for creating probe arrays adapted for various tasks, such as de novo sequencing or re-sequencing are described in U.S. patent application Ser. No. 08/510,521, filed Aug. 2, 1995 and International application PCT/US94/12305, filed Oct. 26, 1994.

The length of an oligonucleotide probe segment can vary to accommodate the sensitivity of the detection system and its ability to discriminate between positive and negative hybridization signals. Accordingly, the length of the oligonucleotide probe segment can be increased to accommodate weaker detection signals or poorly hybridizing target nucleic acids. In general, for example, there can be about 50 oligonucleotide segments per centimeter to about one million oligonucleotide segments per centimeter. Preferably, there are at least about 100, more preferably 200, oligonucleotide segments per centimeter. Even more preferably, there are at least about 500 oligonucleotide segments per centimeter. Most preferably, there are at least about 1000 oligonucleotide segments per centimeter. However, in some especially preferred embodiments there are at least about ten thousand, or even about one hundred thousand, oligonucleotide segments per centimeter.

Preparation of Target Samples

The target polynucleotide is any nucleic acid from any source. For example, the target nucleic acid can be genomic DNA, cDNA, RNA that is unpurified, purified or partially purified. The source can be bacterial, plant or animal. The source can be a cell culture of bacterial, plant, mammalian or yeast cells. The source can be any tissue isolated from a test subject (except exclusively red blood cells). For example, target samples can be whole blood, peripheral blood lymphocytes or PBMC, skin, hair, or semen or other clinical or forensic sample. The source can be saliva, mucus, serum, urine or other body fluid, particularly if one is interested in bacterial or viral nucleic acids. If the target is mRNA, the sample is obtained from a tissue in which the mRNA is expressed. If the target polynucleotide in the sample is RNA, it can be reverse transcribed to DNA. DNA samples, or cDNA resulting from reverse transcription, can be amplified, e.g., by polymerase chain reaction (PCR), before use. Depending on the selection of primers and amplifying enzyme(s), the amplification product can be RNA or DNA. Paired primers are selected to flank the borders of a target polynucleotide of interest. More than one target can be simultaneously amplified by multiplex PCR in which multiple paired primers are employed.

The target can be labeled at one or more nucleotides before hybridization in the microcapillary hybridization chamber. Such labeling can, for example, be done during amplification. For some target polynucleotides (depending on size of sample), e.g., episomal DNA, sufficient DNA is present in the tissue sample to dispense with the amplification step.

Preferably, the detectable label is a luminescent label. Useful luminescent labels include fluorescent labels, chemi-luminescent labels, bio-luminescent labels, and calorimetric labels, among others. Most preferably, the label is a fluorescent label such as a fluorescein, a rhodamine, a polymethine dye derivative, a phosphor, and so forth. Commercially available fluorescent labels include, inter alia, fluorescein phosphoramidites such as Fluoreprime (Pharmacia, Piscataway, N.J.), Fluoredite (Millipore, Bedford, Mass.) and FAM (ABI, Foster City, Calif.).

Useful light scattering labels include large colloids, and especially the metal colloids such as those from gold, selenium and titanium oxide. Radioactive labels can also be used, for example, “P. This label can be detected by a phosphoimager. Detection, of course, depends on the resolution of the imager. Phosophoimagers are available having resolution of 50 microns. Accordingly, this label is currently useful when segments are at least that size.

When the target strand is prepared in single-stranded form as in preparation of target RNA, the sense of the strand should of course be complementary to that of the probes on the chip. This is achieved by appropriate selection of primers.

The target is preferably fragmented before placement in the microcapillary hybridization chamber to reduce or eliminate the formation of secondary structures in the target. The average size of targets segments following hybridization is usually larger than the size of oligonucleotide probes in the chamber.

Hybridization Assays

The microcapillary hybridization chambers of the invention can be used in any hybridization procedure known to one of skill in the art. For example, the microcapillary hybridization chambers can be designed to provide probes specific for analyzing gene expression, genetic polymorphisms, single nucleotide polymorphisms (SNP), disease management, and the like.

Gene expression arrays can be made up of probes complementary to expressed sequence tags (ESTs) from any species or mammalian of interest can be used. Gene expression arrays can also contain probes corresponding to a number of reference and control genes. These reference and control standards make it possible to normalize data from different experiments and compare multiple experiments on a quantitative level.

Genetic polymorphism analysis arrays can be made up of probes complementary to any known deletion, insertion, substitution or other variation in a nucleic acid sequence of interest. Such arrays enable researchers to identify the base present at specific sequence locations and map differences in the thousands of genes that make up the human genome. Polymorphisms in any sequenced nucleic acid can be detected. After the sequences are determined for the first time, it becomes increasingly valuable to identify polymorphisms (or variations) in these genes and to understand how these polymorphisms impact biological function and disease. Such associations can be made using DNA samples from many affected and unaffected individuals for each disease under study.

Single Nucleotide Polymorphism (SNP) mapping assays accelerate genetic analysis of polymorphisms by minimizing labor, data analysis time and total time required to run complex genotyping studies. The mapping assays enable study of the links between polymorphisms and disease, the mechanisms that lead to disease, and patient response to treatment.

Disease management involves analysis of gene expression profiles and polymorphisms that correlate with a specific disease or therapeutic response. Rapid and accurate analysis of this genetic information can facilitate diagnosis and disease management. For example, the microcapillary hybridization chambers of the invention permit diagnosis and disease management of infectious disease, cancer and drug metabolism. Information obtained from the use of the present microcapillary hybridization chambers enables physicians and researchers to understand the genetic basis and progression of disease and patient response to treatment. Microcapillary hybridization chambers of the invention can be used to correlate specific mutations with patient outcomes under varied therapeutic drug regimes. With data gathered through the present methods, scientists can develop more detailed prognoses, drug therapies and treatment strategies. efficient and simultaneous analysis of multiple genotypes associated with drug metabolism defects.

Hybridization assays in the present microcapillary hybridization chambers involve a hybridization step and a detection step. In the hybridization step, a hybridization mixture containing the target and other reagents such as denaturing agents or renaturation accelerants are brought into contact with the linear array of probes in the microcapillary chamber and incubated at a temperature, and under conditions, and for a time appropriate to allow hybridization between the target and any complementary probes. Usually, unbound target molecules are then removed from the array by washing with a wash mixture that does not contain the target, such as hybridization buffer. Washing substantially removes any unbound target molecules in the chamber, effectively leaving only target molecules bound to the probes in the chamber. In the detection step, the probes to which the target has hybridized are identified. Because the nucleic acid sequence of the probes at each probe segment location is known, identifying the locations at which target has bound provides information about the identity and sequences of hybridized target nucleic acids.

Assays using the present microcapillary hybridization chambers generally involve contacting an oligonucleotide array with a sample under the selected reaction conditions, optionally washing the array to remove unreacted molecules, and analyzing the biological array for evidence of reaction between target molecules the probes. These steps involve handling fluids.

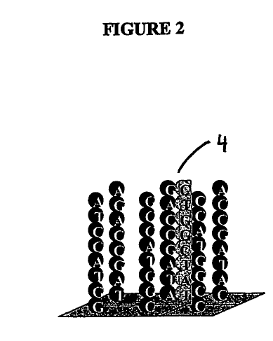

For example, FIG. 2 illustrates the interaction of a target probe system in accordance in a typical chip system. In FIG. 2 the probes are oligonucleotides and the targets are the complementary sequences of the oligonucleotide probes. In FIG. 2, six different probes are shown. Each square in FIG. 2 represents a different probe. Probe 4 is complexed with its complimentary oligonucleotide sequence.

In the present invention probes are attached to the bore region of the tubing in bands or segments. Each segment represents a discrete location of a specific type of probe.

These steps can be automated using automated fluid handling systems. A robotic device can be programmed to set appropriate reaction conditions, such as temperature, add reagents to the chamber, incubate the chamber for an appropriate time, remove unreacted material, wash the chamber, add reaction substrates as appropriate and perform detection assays. The particulars of the reaction conditions are chosen depends upon the purpose of the assay, the probe type, the label type and the target concentration.

Conditions sufficient to permit hybridization include salt, pH, denaturant-renaturant and target concentrations that allow detectable hybridization between probe and target nucleic acids. The hybridization mixture can include the target nucleic acid molecule in an appropriate solution, i.e., a hybridization buffer. The target nucleic acid molecule is present in the mixture at a concentration between about 0.005 nM target per ml hybridization mixture and about 50 nM target per ml hybridization mixture, preferably between about 0.5 nM/ml and 5 nM/ml or, more preferably, about 1 nM/ml and 2 nM/ml. The target nucleic acid molecule preferably includes a detectable label, for example, a fluorescent, phosphorescent or radioactive label.

Denaturing agents can also be included in the hybridization solution. As used herein, the term “denaturing agent” refers to compositions that lower the melting temperature of double stranded nucleic acid molecules by interfering with hydrogen bonding between bases in a double stranded nucleic acid or the hydration of nucleic acid molecules. Denaturing agents can be included in hybridization buffers at concentrations of about 1 M to about 6 M and, preferably, about 3 M to about 5.5 M. Denaturing agents include formamide, formaldehyde, DMSO (“dimethylsulfoxide”), tetraethyl acetate, urea, guanidium thiocyanide (GuSCN), glycerol and chaotropic salts. As used herein, the term “chaotropic salt” refers to salts that function to disrupt van der Waal's attractions between atoms in nucleic acid molecules. Chaotropic salts include, for example, sodium trifluoroacetate, sodium tricholoroacetate, sodium perchlorate, guanidine thiocyanate (GuSCN), and potassium thiocyanate. GuSCN preferably is used at 2 M, and can be used at concentrations up to at least about 5M.

As used herein the term “renaturant” or “renaturation accelerant” refers to compounds that increase the speed of renaturation of nucleic acids by at least 100-fold. They generally have relatively unstructured polymeric domains that weakly associate with nucleic acid molecules. Accelerants include heterogenous nuclear ribonucleoprotein (“I RP”) Al and cationic detergents such as, preferably, CTAB (“cetyltrimethylammonium bromide”) and DTAB (“dodecyl trimethylammonium bromide”), and, also, polylysine, spermine, spermidine, single stranded binding protein (“SSB”), phage T4 gene 32 protein and a mixture of ammonium acetate and ethanol. While not wishing to be limited by theory, renaturation accelerants appear to speed up renaturation by creating multi-step association reactions with reduced rates of dissociation of a highly dynamic encounter complex and provide an orientation-independent free energy of association, and create a new transition state that is less changed in translation and rotational entropy with respect to the reactants. B. W. Pontius, “Close encounters: why unstructured, polymeric domains can increase rates of specific macromolecular association,” TIBS May 1993 pp 181-186. Renaturation accelerants can be included in hybridization mixtures at concentrations of about 1 ˜tM to about 10 mM and, preferably, 1 mM to about 1 mM. The CTAB buffers work well at concentrations as low as 0.1 mM.

A variety of hybridization buffers are useful for the hybridization assays of the invention. By way of example, but not limitation, the buffers can be any of the following:

3 M TMACI, 50 mM Tris-HCI, 1 mM EDTA, 0.1% N-Lauroyl-Sarkosine (NLS);

2.4 M TEACI, 50 niM Tris-HCI, pH 8.0, 0.1% NLS;

1 M LiCl, 10 mM Tris-HCI, pH 8.0, 10% Formamide;

2 M GuSCN, 30 mM NaCitrate, pH 7.5;

1 M LiCl, 10 mM Tris-HCI, pH 8.0, 1 mM CTAB;

0.3 mM Spermine, 10 mM Tris-HCI, pH 7.5; or

2 M NH₄OAc with 2 volumes absolute ethanol.

Addition of small amounts of ionic detergents (such as N-lauroyl-sarkosine) are useful. LiCl is preferred to NaCI.

Additional examples of hybridization conditions are provided in several sources, including: Sambrook et al., Molecular Cloning. A Laboratory Manual (1989), 2nd Ed., Cold Spring Harbor, N.Y.; and Berger and Kimmel, “Guide to Molecular Cloning Techniques,” Methods in Enzymology, (1987), Volume 152, Academic Press, Inc., San Diego, Calif.; Young and Davis (1983) Proc. Natl. Acad. Sci. (U.S.A.) 80: 1194.

The hybridization mixture is placed in contact with the array and incubated. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C. or about 65° C. For example, for probes of about 14 nucleotides hybridization is usually at about 37° C. to 45° C. For probes longer than about 14 nucleotides, about 37° C. to about 45° C. is preferred. For shorter probes, about 55° C. to about 65° C. is preferred.

The target is incubated with the probe array for a time sufficient to allow the desired level of hybridization between the target and any complementary probes in the array. The time can vary somewhat with the temperature and concentration of target used during hybridization. The time for hybridization can vary from, for example, about thirty seconds to about twenty-four hours, preferably about one minute to about sixteen hours, more preferably about fifteen minutes to about eight hours. Using a hybridization temperature of 25° C. a clear signal is obtained in at least about 30 minutes to two hours.

After incubation with the hybridization mixture, the array usually is washed, for example, first with the hybridization buffer and/or then with a solution containing small amounts of detergent and lower salt concentrations. Then the array can be examined to identify the probes to which the target has hybridized.

Detecting Hybridization

In the detection step, the probes to which the target has hybridized are identified. Because the nucleic acid sequence of the probes at each probe segment location is known, identifying the locations at which target has bound provides information about the particular sequences of these targets. In general, hybridization is detected either because each target molecule is labeled and signal is detectable only at locations or probe-target hybrids or because a label can discriminate between double-stranded probe-targets and single-stranded probes, binding only to the double-stranded probe-targets.

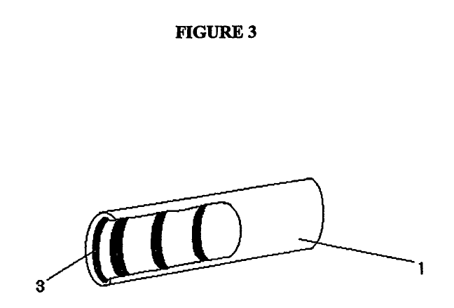

FIG. 3 illustrates a schematic diagram of the microcapillary hybridization chamber showing discrete segments where hybridization has occurred. FIG. 3 shows a microcapillary hybridization chamber where a portion of the chamber has been cut away to review more of the bore region 2 of the tube where the probes are located. Distinct bands or segments of probes that have been hybridized can be seen as circumferential rings 3.

Signal generated from a detectable label in the microcapillary chamber can be detected manually or by using an automated detection device or reader. The nature of the detection device used depends upon the particular type of label attached to the target molecules. In one embodiment the detection device can hold and sequencially move the capillary tubing of the present microcapillary chambers through a sensor chamber that reads the signal emitting from the labels attached to the hybridized target or the hybridized target-probes.

For example, a fluorescent label can be attached to each target molecule or can be attached to a reporter molecule that recognizes and binds to double-stranded target-probes. Excitation radiation, from an excitation source having a first wavelength, can be projected into the microcapillary hybridization chamber(s). Such excitation radiation excites the fluorescent label or reporting molecules on a segment of the microcapillary hybridization chamber. The label or reporter molecule then emits radiation that has a wavelength that is different from the excitation wavelength. Collection optics can then collect the emission from the sample and image it onto a detector. The detector generates a signal proportional to the amount of radiation sensed thereon. The signals can be assembled to represent an image associated with the plurality of segments from which the emission originated.

According to one embodiment, a multi-axis translation stage moves the microcapillary tubing of the hybridization chamber to position different segments for scanning. As a result, a one-dimensional image of the oligonucleotide probe array is obtained.

The oligonucleotide array reader can include an auto-focusing feature to maintain the sample in the focal plane of the excitation light throughout the scanning process. Further, a temperature controller may be employed to maintain the sample at a specific temperature while it is being scanned. The multi-axis translation stage, temperature controller, auto-focusing feature, and electronics associated with imaging and data collection can be managed by an appropriately programmed digital computer.

The detection device also can include a line scanner, as described in U.S. Pat. No. 5,578,832. Excitation optics focuses excitation light to a line at a sample, simultaneously scanning or imaging a strip of the sample. Surface-bound fluorescent labels from the array fluoresce in response to the light. Collection optics image the emission onto a linear array of light detectors. By employing confocal techniques, substantially only emission from the light's focal plane is imaged. Once a segment has been scanned, the data representing the one-dimensional image can be stored in the memory of a computer. According to one embodiment, a multi-axis translation stage moves the device at a constant velocity to continuously integrate and process data. Alternatively, galvometric scanners or rotating polyhedral mirrors may be employed to scan the excitation light across the sample.

The time for detecting an entire oligonucleotide probe array will depend on the size of the array and on the photophysics of the fluorophore (i.e., fluorescence quantum yield and photodestruction yield), as well as on the sensitivity of the detector.

A computer can transform the data into another format for presentation. Data analysis can include the steps of determining, e.g., fluorescent intensity as a function of capillary segment position from the data collected, removing “outliers” (data deviating from a predetermined statistical distribution), and calculating the relative binding affinity of the targets from the remaining data. The resulting data can be displayed as an image with color in each region varying according to the light emission or binding affinity between targets and probes therein.

The amount of binding at each segment of the microcapillary chamber can be determined during hybidization, for example, at several time points after the targets are contacted with the array. The amount of total hybridization can be determined as a function of the kinetics of binding based on the amount of binding at each time point. Thus, it is not necessary to wait for equilibrium to be reached. The dependence of the hybridization rate for different oligonucleotides on temperature, sample agitation, washing conditions (e.g., pH, solvent characteristics, temperature) can easily be determined in order to maximize the conditions for rate and signal-to-noise. Alternative methods are described in Fodor et al., U.S. Pat. No. 5,324,633, incorporated herein by reference.

The dependence of the hybridization rate of different oligonucleotide probes on temperature, sample agitation, washing conditions (e.g., pH, solvent characteristics, temperature) can easily be determined in order to maximize the conditions for rate and signal-to-noise.

Electric Field Stringency Control: An Example.

According to another aspect of the present invention, high specificity between a probe and a target can also be obtained by application of an electrical potential to the microcapillary tube. An electrical potential can be used alone or in combination with more traditional stringency conditions as discussed above and, for example, at least one of temperature, salts, detergents, solvents, chaotropic agents, denaturants and combinations thereof. According to this embodiment an electrical potential or field is applied either to the entire microcapillary tube or to each band separately.

DNA and nucleic acid fragments typically have a net negative charge. If a single electric potential is applied to the microcapillary tube the positive end of the potential will be at the end of the microcapillary tube opposite the end where the sample to be analyzed, or targets, is loaded. The positive end of the potential will draw the nucleic acid fragments through the tube allowing them to interact with the probes fixed in each band. After the fragments have hybridized to some degree with their associated probes the electrical potential applied to the microcapillary tube can be adjusted to remove fragments that are not full complements, and thus may act as false compliments, such that a high level of stringency is achieved.

In a further embodiment, a range of electrical potentials can be scanned through for each probe band. After the desired range of electrical potentials is analyzed the electrical potential can be adjusted such that denaturation of the hybridized probe-target complex, or specific binding entity, occurs. After the denaturation of the hybridized complexes has occurred the microcapillary tube may be reused.

Alternatively, each probe band may have its own electrical potential associated with it in which it hybridizes, under high stringency, with its compliment and nothing else. These individual electrical potentials can be the same as other probe bands or different from each other. Applying a unique electrical potential to each probe band allows for different levels of stringency to be applied to the same microcapillary tube instead of a single stringency level at any one time as is the case when a single electrical potential is used or a range of electrical potentials are scanned one electric potential at time.

An illustration of how electronic stringency controls can be allied to the present invention follows. A perfectly matched DNA hybrid is slightly more stable than a mismatched DNA hybrid. By applying an electric potential to the microcapillary tube, either as a single potential applied to the entire tube or unique potentials applied to each probe band, it is possible to denature or remove the mis-matched DNA hybrids while retaining the perfectly matched DNA hybrids. An electrical potential applied to the microcapillary tube can also greatly reduce the time required for hybridization. After analysis of the hybridization of the probe target complexes the electrical potential can be increased above a predefined threshold that causes denaturation of the complexes. The microcapillary tube can then be washed and reused for additional analyses.

In general, it should be emphasized that the various components of embodiments of the present invention can be implemented in hardware, software, or a combination thereof. In such embodiments, the various components and steps would be implemented in hardware and/or software to perform the functions of the present invention. Any presently available or future developed computer software language and/or hardware components can be employed in such embodiments of the present invention. For example, at least some of the functionality mentioned above could be implemented using C or C++ programming languages.

Thus, it is seen that systems and methods for making and use a microcapillary hybridization chamber are provided. One skilled in the art will appreciate that the present invention can be practiced by other than the preferred embodiments which are presented in this description for purposes of illustration and not of limitation and that numerous changes in the details of construction and combination and arrangement of processes and equipment may be made without departing from the spirit and scope of the invention, and the present invention is limited only by the claims that follow. It is noted that equivalents for the particular embodiments discussed in this description may practice the present invention as well.

What is claimed is:

1. A microcapillary hybridization chamber comprising a narrow bore tubing with probe segments, wherein each probe segment comprises oligonucleotide probes covalently attached to the inner wall of the tubing, and wherein the oligonucleotide probes within each segment have identical, known sequences.

2. The microcapillary hybridization chamber of claim 1 with at least 500 probe segments per cm.

3. The microcapillary hybridization chamber of claim 1 with at least 1000 probe segments per cm.

4. A method for performing a hybridization assay between a target nucleic acid molecule and a microcapillary hybridization chamber that comprises:
(a) introducing a hybridization mixture comprising a test sample into the microcapillary hybridization chamber;
(b) incubating the hybridization mixture within the microcapillary hybridization chamber for a time and under conditions sufficient to hybridize a target nucleic acid within the test sample with an oligonucleotide probe attached to the inner wall microcapillary hybridization chamber;
(c) washing unhybridized nucleic acids out of the microcapillary hybridization chamber; and
(d) detecting hybridization between a target nucleic acid and an oligonucleotide probe;
wherein the microcapillary hybridization chamber comprises a narrow bore tubing with probe segments, wherein each probe segment comprises oligonucleotide probes covalently attached to the inner wall of the tubing, and wherein the oligonucleotide probes within each segment have identical, known sequences.

5. The method of claim 4 wherein the target nucleic acid comprises a detectable label.

6. The method of claim 5 wherein the detectable label is a fluorescent molecule.

7. The method of claim 4 wherein the hybridization mixture further comprises a denaturing agent.

8. The method of claim 7 wherein the denaturing agent is formamide, formaldehyde, DMSO, tetraethyl acetate, urea, GuSCN, glycerol or a chaotropic salt.

9. A microcapillary hybridization chamber comprising a narrow bore tubing with probe segments attached to the inner wall of the tubing at predefined positions, wherein each probe segment comprises identical probes.

10. The microcapillary hybridization chamber of claim 1 with at least 500 probe segments per cm.

11. The microcapillary hybridization chamber of claim 1 with at least 1000 probe segments per cm.

12. The microcapillary hybridization chamber of claim 1, wherein each probe segment is selected from deoxyribonucleic acids, ribonucleic acids, synthetic oligonucleotides, antibodies, proteins, peptides, lectins, modified polysaccharides, cells, synthetic composite macromolecules, functionalized nanostructures, synthetic polymers, modified/blocked nucleotides/nucleosides, modified/blocked amino acids, flurophores, chromophores, ligands, chelates, hapten, and combinations thereof.

13. The microcapillary hybridization chamber of claim 1, wherein each probe segment is distinguishable from other probe segments.

14. The microcapillary hybridization chamber of claim 1, wherein each probe segment is immobile.

15. A method for performing a hybridization assay between at least one target and a microcapillary hybridization chamber, said method comprises:
introducing a sample comprising said at lest one target into said microcapillary hybridization chamber;
applying an electrical potential to said microcapillary hybridization chamber with said at least one target;
washing unhybridized said at least one target out of said microcapillary hybridization chamber;
detecting hybridization; and
wherein said microcapillary hybridization chamber comprises a narrow bore tubing with probe segments attached to the inner wall of the tubing at predefined positions, wherein each probe segment comprises identical probes.

16. The method according to claim 15, further comprising increasing the electrical potential above a predefined threshold to denature said hybridization.

17. The method according to claim 15, further comprising applying a range of electrical potentials over time to said microcapillary hybridization chamber with said at least one target.

18. The method according to claim 17, further comprising detecting hybridization at each of said electrical potentials.

19. A method of making a microcapillary hybridization chamber comprising:
providing a substrate wall, wherein said substrate wall is internal to a narrow bore capillary tube; and
attaching a plurality of probe segments to said substrate; wherein each of said plurality of probe segments are spatially discrete from each other.

20. The method according to claim 19, wherein attaching said plurality of probe segments to said substrate comprises synthesizing said probes at specific location on said substrate.

21. The method according to claim 20, wherein said plurality of probe segments are oligonucleotides and said oligonucleotides are attached synthesized at said specific locations on said substrate by light-directed oligonucleotide synthesis.

22. A method for controlling the stringency in a microcapillary hybridization chamber comprising applying an electrical potential to each probe segment in said microcapillary hybridization chamber; wherein said microcapillary hybridization chamber comprises a narrow bore tubing with said probe segments attached to the inner wall of the tubing at predefined positions, wherein each of said probe segment comprises identical probes.

23. The method according to claim 22, further comprising detecting the state of hybridization.

24. The method according to claim 22, adjusting each electrical potential until to eliminate mismatched hybridizations.

25. A apparatus for detecting hybridization in a microcapillary hybridization chamber comprising:
a microcapillary hybridization chamber, wherein said microcapillary hybridization chamber comprises a narrow bore tubing with probe segments attached to the inner wall of the tubing at predefined positions, wherein each probe segment comprises identical probes;
a detector for detecting hybridization signals in said microcapillary hybridization chamber; and
a computer system operationally coupled to said detector, the computer system comprises a program comprising:
displaying said detected hybridization signals.

26. The apparatus according to claim 25, wherein said detector comprises excitation optics for focusing excitation light on at least one of said probe segments.

27. The apparatus according to claim 25, wherein said program further comprises determining fluorescent intensity;
removing data outliers; and
calculating the relative binding affinity of said hybridization signals.

28. The apparatus according to claim 25, wherein said program further comprises displaying a image of probe segment colors based on at least one of light emission or binding affinity.
